(19) United States
(12) Patent Application Publication
(10) Pub. No.: US 2023/0405173 A1
(43) Pub. Date: Dec. 21, 2023

(54) AIR REMOVAL LIGHT DEVICE

(71) Applicant: Michael Allan Dolynchuk, Red Deer (CA)

(72) Inventor: Michael Allan Dolynchuk, Red Deer (CA)

(21) Appl. No.: 18/240,687

(22) Filed: Aug. 31, 2023

Related U.S. Application Data
(63) Continuation of application No. PCT/CA2022/050272, filed on Feb. 25, 2022.
(60) Provisional application No. 63/155,066, filed on Mar. 1, 2021.

(30) Foreign Application Priority Data
Mar. 31, 2021 (CA) 3113857

(51) Int. Cl. A61L 9/20; A61B 90/35; F21V 33/00; F21V 21/26

(52) U.S. Cl. CPC A61L 9/20; A61B 90/35; F21V 33/0088; F21V 33/0064; F21V 21/26; A61L 2209/12; F21W 2131/202

(57) ABSTRACT

An air removal light device comprising a movable task light with an air suction channel having an inlet for evacuating air from the area below the task light that is between a practitioner and a patient/client. The device includes a body with an outer edge and bottom surface having at least one lamp. An air suction channel is created around the body by positioning an outer wall around the body, with at least one air inlet located adjacent the bottom surface of the body. The air suction channel has an air outlet which is connected to a vacuum hose and vacuum system to remove air from the area below the light. The removed air is transported through a filter to remove pathogens and particulate matter before being returned to the room or exhausted.

### CROSS-REFERENCE TO RELATED APPLICATIONS

This application is a bypass continuation that claims priority to International Patent Application No. PCT/CA2022/050272 filed on Feb. 25, 2022, which claims priority to Canadian Patent Application No. 3113857 filed on Mar. 31, 2021, and U.S. Provisional Patent Application No. 63/155,066 filed on Mar. 1, 2021, where the entire content of each of the foregoing applications is incorporated herein by reference.

### FIELD OF THE INVENTION

The invention relates to the removal of airborne contaminants, and more specifically to an air removal system incorporated into a light.

### BACKGROUND

Dentists and other dental workers may be subject to high exposure to contaminants and disease-causing pathogens found in the air of dental offices. Many dental procedures routinely create airborne matter, including particulate matter and bioaerosols containing microbes from blood, saliva, and subgingival fluids from a patient. The bioaerosols can contain disease-causing pathogens. At times, a dental worker may wear personal protective equipment (PPE) during a procedure with a patient. However this may not be enough to protect the worker and patient from exposure to airborne pathogens. For example, there may not be an airtight seal between a mask or glasses and the user's face. In addition, aerosols can stay suspended in the air for a prolonged period and may still be present after a user has removed their PPE. Furthermore, a patient does not wear a mask during dental work because of the nature of the work, and therefore the patient is not protected from airborne pathogens. The airborne pathogens can also spread throughout the room and/or dental office and expose people who aren't wearing PPE.

In addition to disease-causing pathogens, the air quality in a dental room may be poor for other reasons. For example, there may be dust and other particulate matter that becomes airborne during dental procedures. This dust and particulate matter can be detrimental to a person's health, especially with chronic exposure.

Dental offices often just rely on a buildings HVAC system for circulating air in the room. This is often not sufficient to remove airborne pathogens like bacteria or viruses. This can lead to less than ideal indoor air quality and may pose increased safety risks during times of a pandemic where pathogens are transmitted through the air.

The inventor has recognized a need to increase air quality and decrease the potential for cross-contamination through airborne pathogens between patients and dental workers. The inventor has also recognized that this need for better and safer practices extends beyond dental workers to surgeons, estheticians, hairdressers, lab technicians, and more.

A review of prior art reveals a number of lighting apparatuses with air flow considerations, including US Publication 2020/0345905; Chinese Publication 202356384; U.S. Pat. No. 4,037,096; Japanese Publication 2005304906; Japanese Patent 9135847; US Publication 2020/0139000; Japanese Publication 2013085878; Japanese Patent 6661092; PCT Publication WO 2019/166114; US Publication 2016/0303271; PCT Publication WO 2020/204661; U.S. Pat. No. 6,551,407. None of these references address the need recognized by the inventor in regard to airborne pathogens.

### SUMMARY

In accordance with the invention, there is provided a light and air removal device comprising a body having an outer edge, a bottom surface and at least one lamp; an outer wall surrounding the outer edge of the body to form an air suction channel around the outer edge of the body, the air suction channel having at least one inlet disposed adjacent the bottom surface of the body; a vacuum hose connected to the air suction channel for connection to a vacuum device; and an adjustable support connected to the body for positioning the body and the at least one inlet to direct light and air suction in an area adjacent the bottom surface of the body.

In some embodiments, the device further comprises an inner wall between the body outer edge and the outer wall, the air suction channel formed between the inner wall and the outer wall.

In some embodiments, the inner wall and the outer wall are parallel to each other. The inner wall and the outer wall may be concentric.

In some embodiments, the at least one inlet surrounds an outer periphery of the bottom surface of the body. The at least one inlet may be an annular inlet and the outer periphery may be circular shaped.

In some embodiments, the device further comprises at least one secondary air suction inlet located on the bottom surface of the body and connects to the vacuum hose via a secondary air suction channel.

In some embodiments, the device further comprises a handle connected to the body for moving the device.

In some embodiments, the adjustable support is a swing arm.

In some embodiments, the air suction channel is a hollow ring surrounding the body having holes as air inlets.

In some embodiments, the at least one lamp is suitable for use as any one of the following: a dental light, a surgical light, an aesthetician light and a hair stylist light.

In some embodiments, the device includes a UV lamp. The UV lamp may be a LED UV rope light that is positioned around the outer edge of the body.

In one aspect of the invention, there is provided a kit for retrofitting a light to include air suction comprising a shroud for placement around a body of the light to form an air suction channel and at least one air suction inlet adjacent an outer periphery of a bottom surface of the body, the air suction channel having an outlet for connection to a vacuum hose and a vacuum device.

In some embodiments, the device further comprises the vacuum hose for connection to the outlet and to the vacuum device.

In some embodiments, the shroud is friction fit to the light body.

In some embodiments, the kit includes at least one attachment mechanism for attaching the shroud to the light body.

In some embodiments, the kit further includes a LED UV rope light for placement around the body of the light.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention. Similar reference numerals indicate similar components.

FIG. 1 is a front perspective view of the air removal light device in accordance with one embodiment positioned over a dental patient.

FIG. 2A is a front perspective view of one embodiment of the air removal light device.

FIG. 2B is a bottom view of the air removal light device of FIG. 2A.

FIG. 3 is a flowchart illustrating the flow of air from an area through the air removal light device and through a filter.

FIG. 4 is a bottom view of one embodiment of the air removal light device.

FIG. 5A is a bottom perspective view of one embodiment of the air removal light device.

FIG. 5B is a side cross sectional view of the air removal light device of FIG. 5A.

FIG. 6A is a side view of one embodiment of the air removal light device comprising a shroud for retrofitting to an existing light device.

FIG. 6B is a bottom view of the shroud of FIG. 6A.

FIG. 7 is a front perspective view of one embodiment of the air removal light device wherein the vacuum body is retrofit onto an existing light device.

FIG. 8A is a side view of an air removal light device comprising an aesthetician's light including a vacuum channel in accordance with one embodiment.

FIG. 8B is a bottom view of the body of the air removal light device of FIG. 8A.

FIG. 9 is a side view of an air removal light device comprising a hanging ring light in accordance with one embodiment.

FIG. 10 is a top perspective view of an air removal light device comprising a hanging device over a restaurant table in accordance with one embodiment.

### DETAILED DESCRIPTION

### Rationale

The inventor has realized the need to decrease the chance of disease transmission between a practitioner and a patient during a procedure. The inventor has accomplished this through the use of an air removal system that evacuates air containing airborne particles and pathogens directly from the area between the practitioner and the patient. The system limits the spread of the airborne particles and pathogens since they are removed almost immediately after they are emitted into the air from the patient's mouth or nose, instead of allowing the airborne particles and pathogens to move around the room towards other people and/or remain suspended in the air. The inventor has accomplished this in a cost-effective and efficient way that minimizes equipment requirements by using a vacuum shroud that can be built in or added onto existing task lighting that a practitioner would use anyway.

Task lighting devices are standard fixtures in dental, surgical and esthetician rooms to provide the desired illumination on an area that is being examined or worked on. The lighting devices are usually specialty devices having the desired intensity, color temperature, illumination pattern and other features. The lighting devices are usually movable, for example by use of a swing arm, so that the user can direct the light to illuminate the desired area. The lighting device may be mounted to a ceiling, cabinet, rail or other feature in a room. It may also be placed on a floor, table or other surface.

### INTRODUCTION

Various aspects of the invention will now be described with reference to the figures. For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that, according to the knowledge and judgment of persons skilled in the art, such alternative features may be substituted in various combinations to arrive at different embodiments of the present invention.

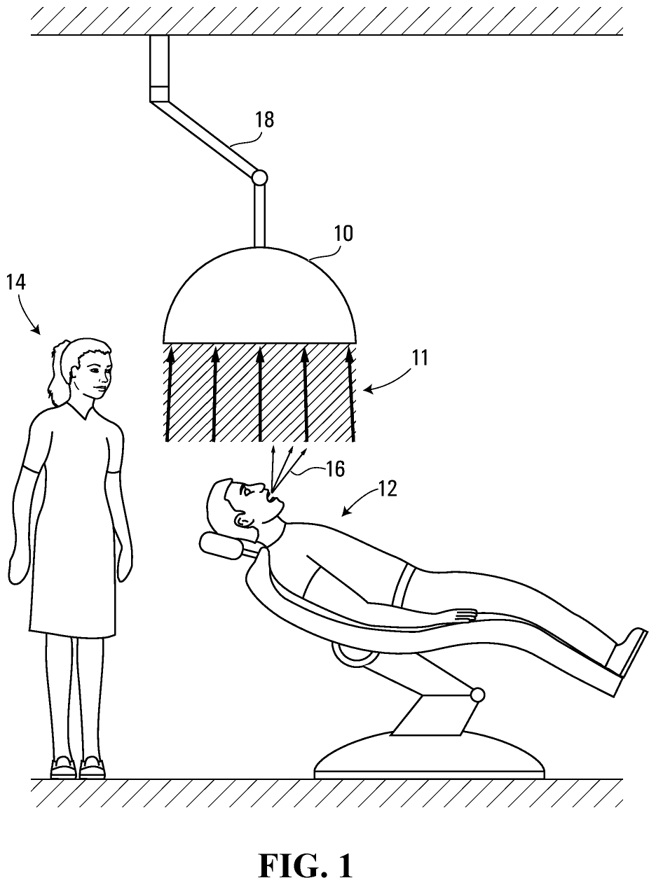

With reference to the figures, an air removal light device 10 described. As shown in FIG. 1, the air removal light device 10 is positioned to evacuate air from an area 11 between a patient 12 and a practitioner 14. In this case, airborne matter 16 containing bioaerosols and particulate matter from around the patient's mouth and nose are vacuumed up into the air removal light device 10. The air removal light device 10 is movable, such as by a swing arm 18, in order to position the device where it as needed. It is connected to a vacuum system (not shown) that can be turned on to evacuate the air from area 11. The air removal light device 10 also provides lighting to illuminate the area on the patient 12 that the practitioner 14 needs to see.

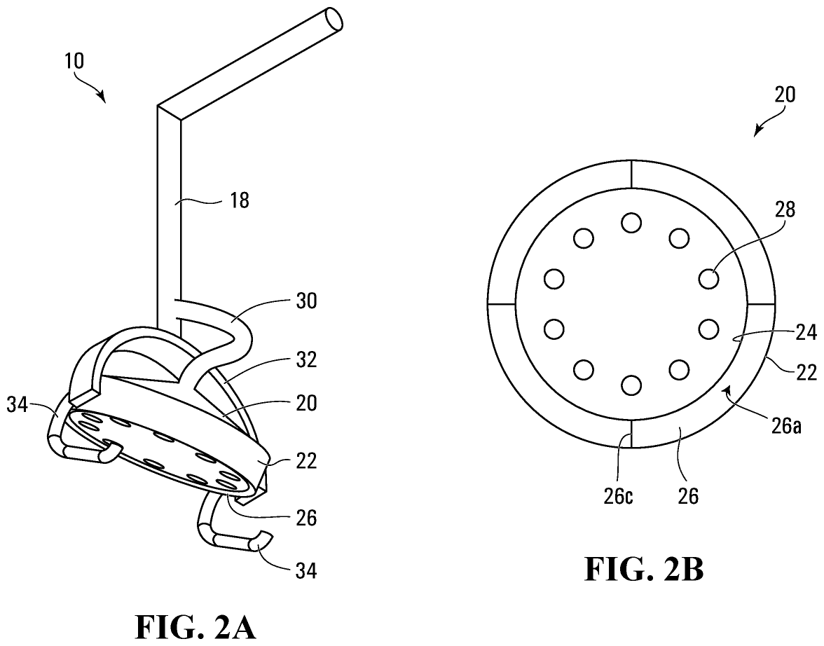

One embodiment of the device 10 is shown in FIGS. 2A and 2B. The device 10 includes a body 20 having an outer wall 22 and an inner wall 24 defining an air suction channel 26 around the outer circumference of the body 20 with an inlet 26a adjacent the bottom surface 20a of the body 20. The body bottom surface 20a includes one or more lamps 28 for illuminating the area 11 that the body bottom surface 20a is facing. The air suction channel 26 is connected to a vacuum hose 30 that runs through the swing arm 18 to connect to a vacuum system (not shown) for evacuating air from the area 11 adjacent the body bottom surface 20a. The body 20 is connected to a support 32, which is connected to the swing arm 18. The body 20 can pivot with respect to the support 32 and can move in multiple directions by way of the swing arm 18 to position the light and air suction where it is needed. The body includes one or more handles 34 for a practitioner to grasp to move the device 10. The air suction channel 26 may include one or more ribs 26c, shown in FIG. 2B, for spacing and support of the channel.

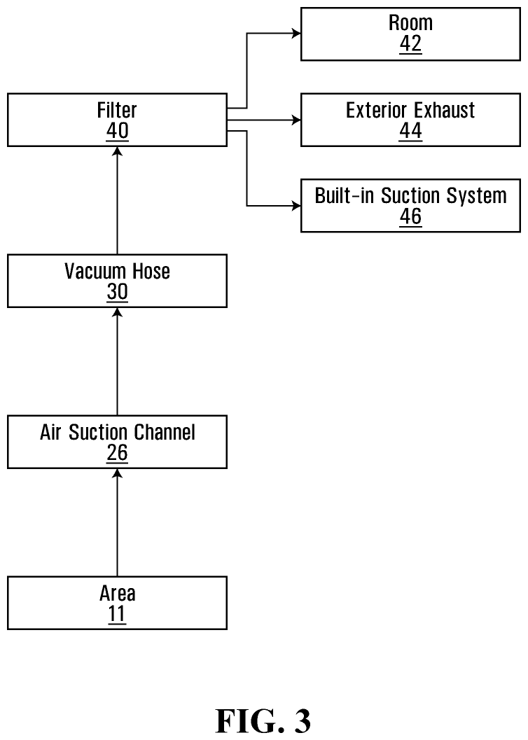

FIG. 3 illustrates the flow of air during the process. Contaminated air from the area 11 between the patient and practitioner flows into the air suction channel 26 of the device 10, through the vacuum hose 30 and through a filter 40. The filtered air can then be directed in a number of ways, including being returned back into the room 42, exiting the building through an exterior exhaust 44, and/or being disposed into an existing built-in suction system 46, in which case the air likely flows into a sewer line.

The filter 40 can be a number of different types of filters, including a HEPA filter, UV filter, a water filter, or any other suitable filter that can remove airborne pathogens and/or other matter.

The device 10 can be made as an integral part of a lighting device that is typically used by a practitioner. In this case, the lighting device includes an air suction channel around the outer border of the light body that is connected to a vacuum hose. The vacuum hose can be inside the structure of the device, for example it can run through the inside of the arm from the body and connect to a vacuum system at the other end of the arm or further away.

Alternatively, the system can be built as an add-on to retrofit an existing lighting device. In this case, a shroud comprising an inner wall and outer wall is configured to be attached to the lighting device such that the shroud surrounds the body to create the air suction channel around the lighting device. The air suction channel includes an inlet in which air enters, and an outlet attached to a vacuum hose and vacuum system to suck air in from the area between the patient and practitioner and move it through a filter to clean the air of pathogens and particulate matter.

A number of light designs with a variety of purposes can be adapted to incorporate an air suction channel to evacuate air in the area between a practitioner and patient/client. The air removal light device can be used by various practitioners, including dentists, surgeons, estheticians, lab technicians, hairdressers and more. Some example embodiments are illustrated in the figures described below.

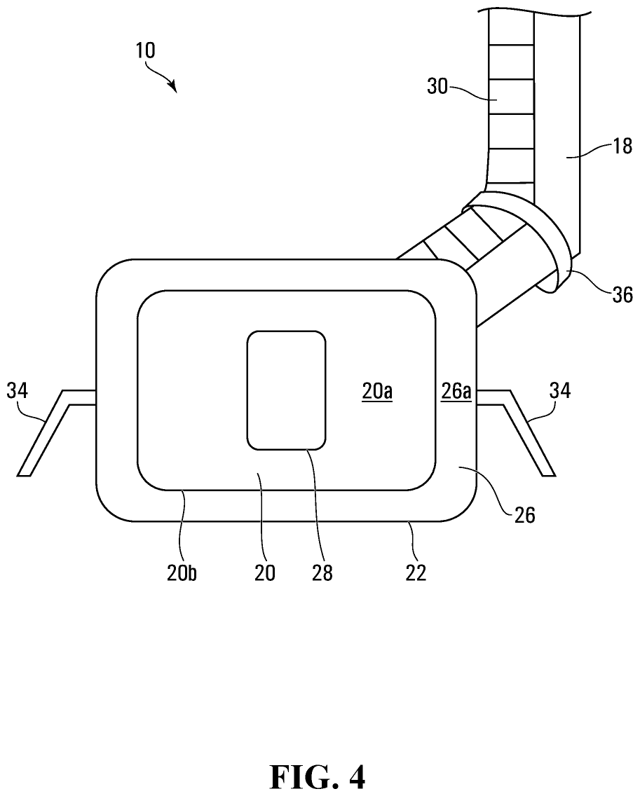

FIG. 4 illustrates one embodiment of the air removal light device 10 that uses a lighting device similar to what dentists and surgeons may use. In this embodiment, the border of the body bottom surface 20a is substantially rectangular shaped and includes an outer wall 22 to create the air suction channel 26 around the outer edge of the body bottom surface 20a. In this case, the outer edge 20b of the bottom body surface 20a forms the inner wall 24 of the air suction channel 26. In this drawing, the vacuum hose is shown as removably attached to the swing arm 18 via a fastener 36. Alternatively, the vacuum hose 30 could run through the inside of the swing arm 18. If so, an access panel (not shown) can be located on the outer surface of the swing arm 18 to allow for easy removal and cleaning of the vacuum hose 30.

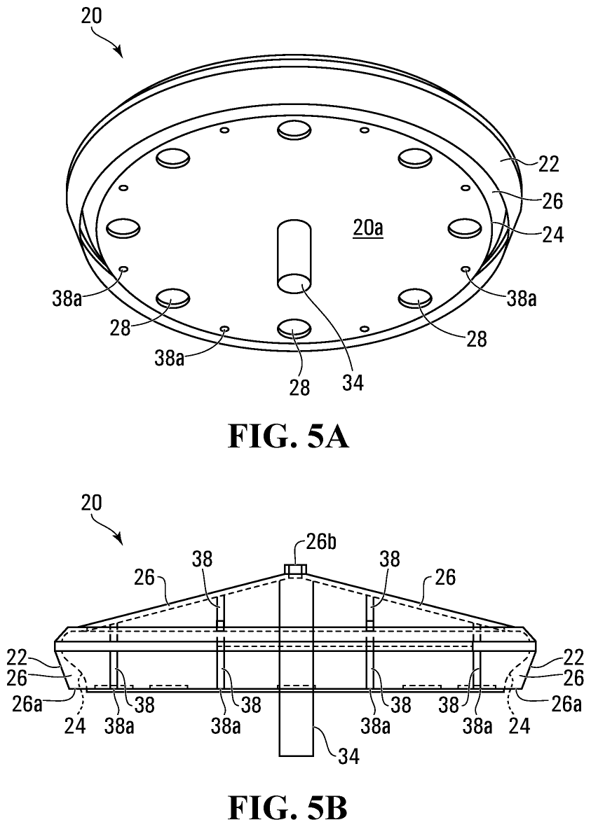

FIGS. 5A and 5B illustrate one embodiment of the air removal light device 10 that uses another style of dental/surgical light. In this embodiment, the inlet 26a of the air suction channel 26 is an annular inlet positioned around the circumference of the circular shaped bottom surface 20a of the body 20, such that the inlet 26a is adjacent the bottom surface 20a. Secondary air suction channels 38 are spaced apart through the body 20 and have secondary inlets 38a in the bottom surface 20a of the body 20 to provide for additional air suction. As shown in FIG. 5B, the secondary air suction channels 38 connect to the air suction channel 26 which has an outlet 26b that connects to the vacuum hose (not shown). In this embodiment, the handle 34 for positioning the device is a post-style handle extending downwardly from the bottom surface 20a of the device. The handle 34 can be removed for easy cleaning.

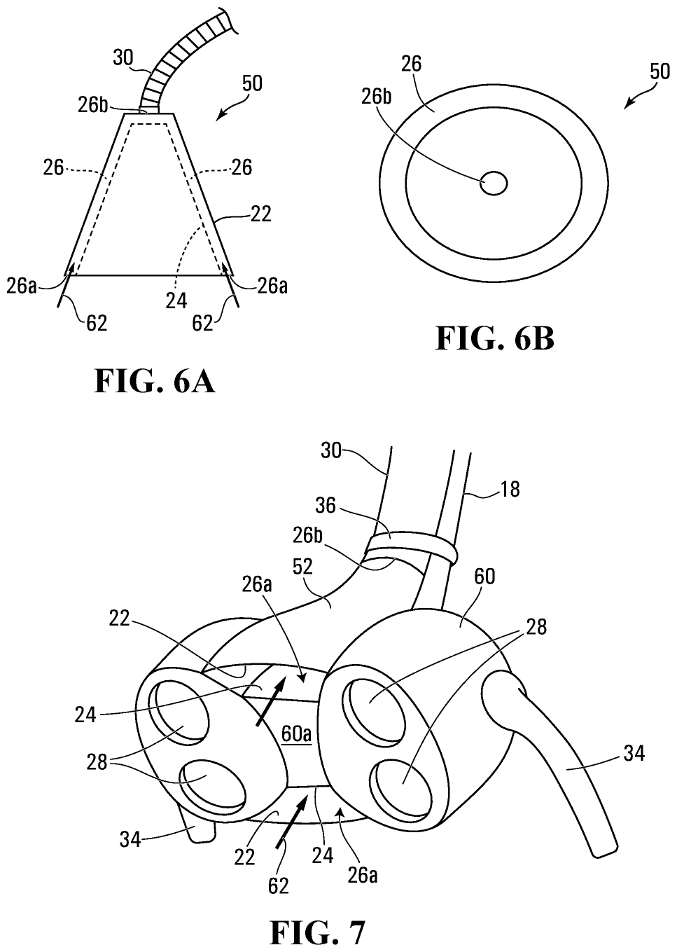

FIGS. 6A and 6B illustrate an embodiment comprising a retro-fit vacuum shroud with an outer wall 22 and inner wall 24 forming an air suction channel 26 having an inlet 26a and outlet 26b, the outlet connectable to a vacuum hose 30. In this case, the shroud attaches around an existing lighting device to turn the lighting device into an air removal light device. The shroud may include attachment mechanisms, like clips, or may be friction fit around the lighting device.

FIG. 7 illustrates one embodiment wherein a vacuum body 52 is connected to an irregular shaped lighting device 60. The vacuum body 52 includes an outer wall 22 and inner wall 24 that create an air suction channel having an inlet 26a adjacent the bottom surface 60a of the lighting device to evacuate air 62 from the area adjacent the bottom surface 60a of the lighting device. The vacuum body 52 includes a clip 36 for fastening the body to the lighting device. A vacuum hose 30 connects to the air suction channel outlet 26b to connect to a vacuum system.

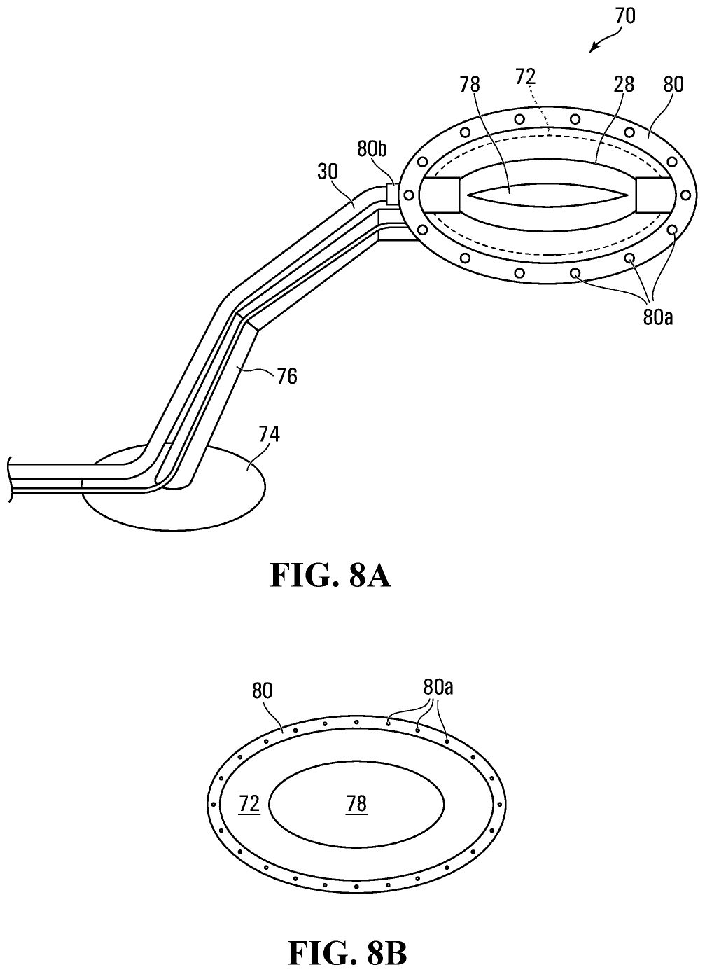

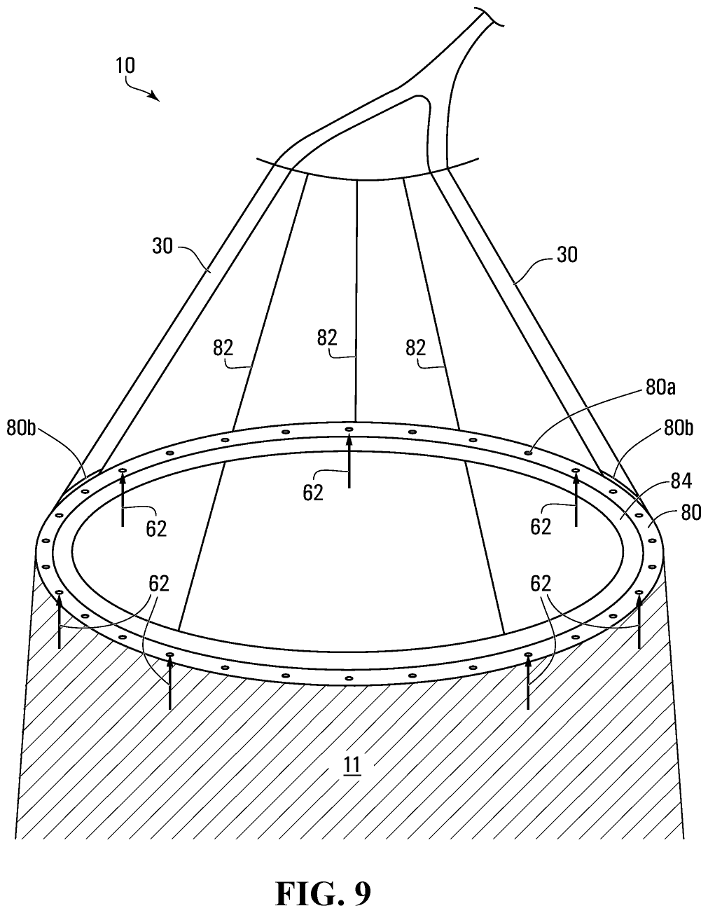

FIGS. 8A and 8B illustrate one embodiment that uses a typical estheticians lighting device 70 that includes a light body 72 with lamps 28 and a magnifying glass 78. The lighting device 70 has a base 74 that sits on a table and has a support arm 76 through which the electrical wires run from the base 74 to the light body 72. A hollow ring 80 forming the air suction channel is positioned around the perimeter of the light body 72 and has holes 80a that form the air suction inlets. An outlet 80b is formed in the hollow ring 80 that connects to the vacuum hose 30. The vacuum hose 30 runs adjacent the articulable support arm 76 to the base 74 and beyond, where it eventually connects to a vacuum system.

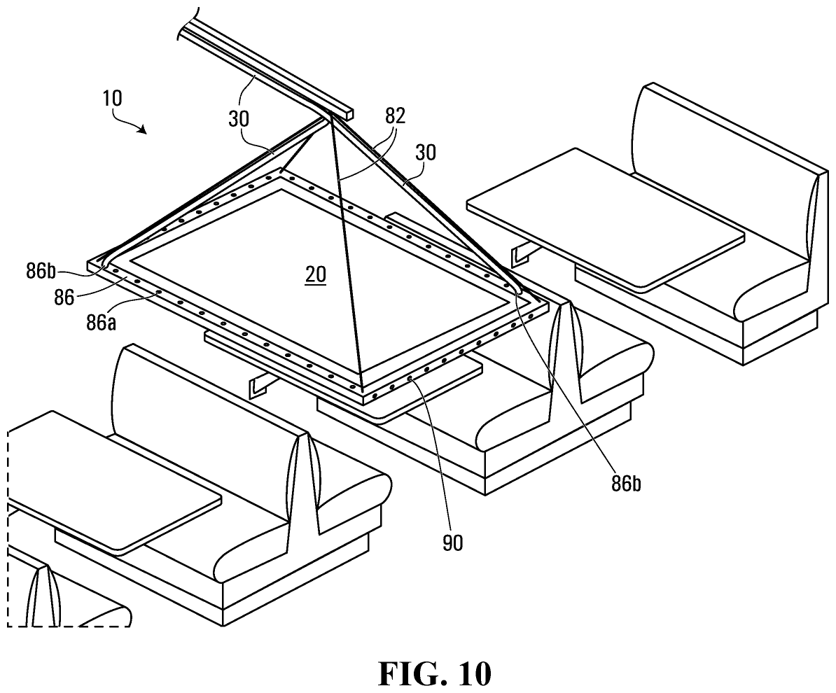

FIG. 9 illustrates one embodiment of the air removal light device 10 that uses a circular pendant light. In this embodiment, the circular pendant light hangs from the ceiling via wires 82 over an area in which a patient/client is located, for example a client sitting in a chair at a hair salon or at a table. The device includes an LED circular bulb channel 84 that directs light downward. Around the circumference of the bulb channel 84 there is a hollow ring 80 that forms the air suction channel and has inlets 80a spaced around the bottom surface of the hollow ring 80 to evacuate air 62 from the area 11 between the client and the practitioner. One or more vacuum hoses 30 connect to one or more air suction outlets 80b on the hollow ring 80 at one end, and to a vacuum system at another end to provide air suction through the vacuum hose(s) 30 and hollow ring 80. The height of the air removal light device 10 may be adjustable by adjusting the length of the wires 82 by which the light device is hung.

FIG. 10 illustrates one embodiment of the air removal light device 10 shown suspended over a restaurant table 90 to evacuate air in the area between the device 10 and the table 90. In this embodiment, a vacuum ring 86 comprising a hollow tube surrounds the perimeter of the bottom surface of the device body 20. The hollow tube has holes 86a forming vacuum inlets spaced apart around the bottom surface of the tube. The hollow tube has one or more vacuum outlets 86b which connect to vacuum hose 30. The vacuum hose provides air suction through connection to a vacuum system. The device 10 includes lights to provide lighting over the table.

In some embodiments, the air removal light device includes a UV light that emits UVC wavelengths as well as a regular light. The UV light is preferably an LED light, and is preferably a tube or strip light that surrounds or runs adjacent the edge of the body bottom surface 20a of the air removal light device. The UV light may be built into the air removal light device or can be retrofit to an existing air removal light device or existing light device. The UV light can be turned on to disinfect the area below the light. The UV light can be used between patients/users, since it can be dangerous to a person's skin and eyes. For example, in a dental or surgical room, after the patient has left the room, the practitioner can turn on the UV light and the leave the room. In a short period of time, such as 10 minutes, the area below the light will be sterilized.

The air removal light device can be used for a number of uses, including but not limited to dental rooms, surgical rooms, estheticians, hair salons, restaurants, offices, laboratories (for example, between a lab technician and a sample they are working with), and more.

Although the present invention has been described and illustrated with respect to preferred embodiments and preferred uses thereof, it is not to be so limited since modifications and changes can be made therein which are within the full, intended scope of the invention as understood by those skilled in the art.

1. A light and air removal device, comprising:
a body having an outer edge, a bottom surface, and at least one lamp;
an outer wall surrounding the outer edge of the body to form an air suction channel around the outer edge of the body, the air suction channel having at least one inlet disposed adjacent the bottom surface of the body;
a vacuum hose connected to the air suction channel for connection to a vacuum device; and
an adjustable support connected to the body for positioning the body and the at least one inlet to direct light and air suction in an area adjacent the bottom surface of the body.

2. The device of claim 1, further comprising an inner wall between the body outer edge and the outer wall, the air suction channel formed between the inner wall and the outer wall.

3. The device of claim 2, wherein the inner wall and the outer wall are parallel to each other.

4. The device of claim 2, wherein the inner wall and the outer wall are concentric.

5. The device of claim 1, wherein the at least one inlet surrounds an outer periphery of the bottom surface of the body.

6. The device of claim 5, wherein the at least one inlet is an annular inlet and the outer periphery is circular shaped.

7. The device of claim 1, further comprising at least one secondary air suction inlet located on the bottom surface of the body that connects to the vacuum hose via a secondary air suction channel.

8. The device of claim 1, further comprising a handle connected to the body for moving the device.

9. The device of claim 1, wherein the adjustable support is a swing arm.

10. The device of claim 1, wherein the air suction channel is a hollow ring surrounding the body having holes as air inlets.

11. The device of claim 1, wherein the at least one lamp is suitable for use as any one or combination of the following: a dental light, a surgical light, an aesthetician light, and a hair stylist light.

12. The device of claim 1, wherein the device includes a UV lamp.

13. The device of claim 12, wherein the UV lamp is a LED UV rope light positioned around the outer edge of the body.

14. The device of claim 1, wherein:
the at least one inlet is an annular inlet surrounding an outer periphery of the bottom surface of the body, and
the air suction channel is a hollow ring surrounding the body having holes as air inlets,
the device further comprising:
at least one secondary air suction inlet located on the bottom surface of the body that connects to the vacuum hose via a secondary air suction channel; and
at least one UV lamp.

15. A kit for retrofitting a light to include air suction, the kit comprising:
a shroud for placement around a body of the light to form an air suction channel; and
at least one air suction inlet adjacent an outer periphery of a bottom surface of the body, the air suction channel having an outlet for connection to a vacuum hose and a vacuum device.

16. The kit of claim 15, further comprising the vacuum hose for connection to the outlet and to the vacuum device.

17. The kit of claim 15, wherein the shroud is friction fit to the light body.

18. The kit of claim 15, further comprising at least one attachment mechanism for attaching the shroud to the light body.

19. The kit of claim 15, further comprising a LED UV rope light for placement around the body of the light.
